(19) United States
(12) Patent Application Publication
(10) Pub. No.: US 2017/0102382 A1
(43) Pub. Date: Apr. 13, 2017

(54) ANALYTE TEST STRIP ASSAYS, AND TEST STRIPS AND KITS FOR USE IN PRACTICING THE SAME

(71) Applicant: Becton, Dickinson and Company, Franklin Lakes, NJ (US)

(72) Inventor: James Raymond Petisce, Westford, MA (US)

(21) Appl. No.: 15/126,796

(22) PCT Filed: Jun. 10, 2015
(86) PCT No.: PCT/US15/35169

§ 371 Date: Sep. 16, 2016

Related U.S. Application Data
(60) Provisional application No. 62/029,388, filed on Jul. 25, 2014.

(51) Int. Cl. G01N 33/543

(52) U.S. Cl. CPC G01N 33/54386

(57) ABSTRACT

Methods of evaluating a sample, e.g., a saliva sample, for the presence of an analyte, e.g., glucose, are provided. Aspects of the methods include: placing a sample onto a sample receiving location of a test strip device, where the test strip device includes analyte detection reagents; and then obtaining a signal from the test strip assay device to evaluate the sample for the presence of the analyte; where the methods include contacting the sample with an antibacterial agent at some point during the assay. Also provided are test strips and kits configured for use in the methods.

### CROSS-REFERENCE TO RELATED APPLICATIONS

This application is related to U.S. Provisional Patent Application Ser. No. 62/029,388 filed Jul. 25, 2014, the disclosure of which application is incorporated herein by reference.

### INTRODUCTION

Glycemic maintenance for people afflicted with diabetes is a constant daily burden. At a minimum, effective glycemic maintenance includes monitoring blood glucose levels frequently throughout the day. Glucose monitoring is most frequently accomplished by pricking the fingertip and placing a blood drop onto a glucose measuring strip. Such a method has three fundamental issues. First, pricking the fingertip with a needle, even a small one, is painful, especially when done several times a day. Second, this process is inconvenient and embarrassing to end-users because they usually excuse themselves from the company of other people when performing a finger stick as a matter of courtesy. Lastly, the intrusiveness of the finger stick procedure causes many end-users to forget to perform the procedure when doing so would be most effective, namely before and after each meal at a minimum. Individually or collectively, these three issues are burdensome to, and commonly cause poor compliance by, the end-user.

The use of a saliva sample to measure glucose levels has been attempted unsuccessfully by numerous individuals. Since 1981, about twenty-five clinical studies have been reported in the scientific literature. In these studies, salivary glucose levels were compared to glucose levels made concurrently by capillary finger sticks. Correlation coefficients were too low to report from some studies, such that the investigators merely stated that no correlations existed. Alternatively, when correlation coefficients were reported by investigators, they were poor (r2<0.6).

### SUMMARY

Methods of evaluating a sample, e.g., a saliva sample, for the presence of an analyte, e.g., glucose, are provided. Aspects of the methods include: placing a sample onto a sample receiving location of a test strip device, where the test strip device includes analyte detection reagents; and then obtaining a signal from the test strip assay device to evaluate the sample for the presence of the analyte; where the methods include contacting the sample with an antibacterial agent at some point during the assay. Also provided are test strips and kits configured for use in the methods.

### BRIEF DESCRIPTION OF THE FIGURES

FIG. 1 provides a view of a lateral flow assay test strip device which may be employed in methods according to an embodiment of the invention.

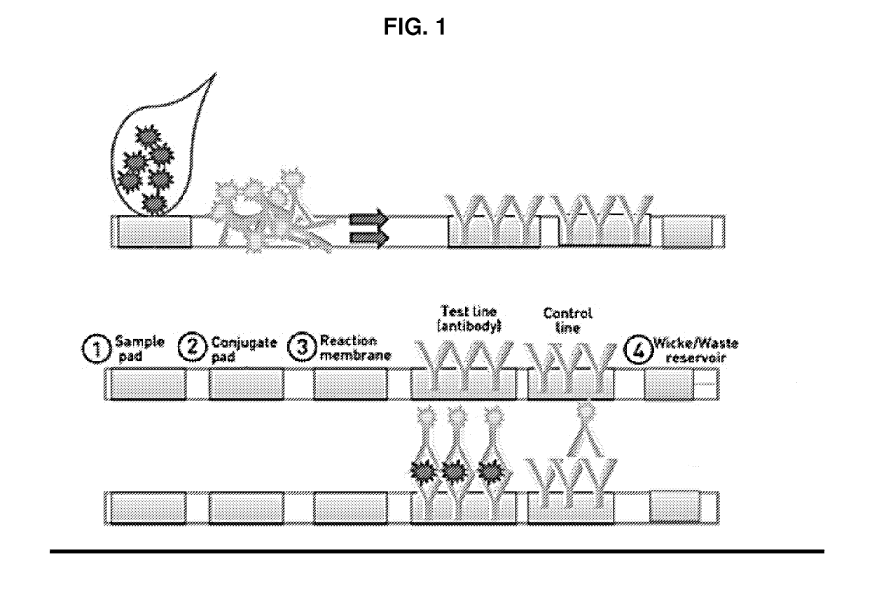

### DETAILED DESCRIPTION

Methods of evaluating a sample, e.g., a saliva sample, for the presence of an analyte, e.g., glucose, are provided. Aspects of the methods include: placing a sample onto a sample receiving location of a test strip device, where the test strip device includes analyte detection reagents; and then obtaining a signal from the test strip assay device to evaluate the sample for the presence of the analyte; where the methods include contacting the sample with an antibacterial agent at some point during the assay. Also provided are test strips and kits configured for use in the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention.

The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, methods will be reviewed first in greater detail, followed by a review of different applications where the methods find use, as well as kits that find use in practicing the methods of the invention.

### Methods

As summarized above, methods of evaluating a sample for the presence of an analyte, e.g., glucose, are provided. Samples of interest include physiological samples, which samples may be saliva, urine, tears, semen, sputum, etc. In some embodiments the sample is a saliva sample. For ease of description the embodiments of the invention are described in terms of the sample being saliva. However, the invention is not so limited. By saliva sample is meant a liquid sample obtained from an oral cavity of a living subject, e.g., a mammal, such as a human. The saliva sample may be employed as is, or pre-processed prior to testing with a test strip, e.g., as described in greater detail below. For example, the saliva sample may be filtered, e.g., to remove crude particles or other material that may be present in the saliva.

Aspects of the methods include assaying the saliva sample with a test strip device to evaluate the sample for presence of the analyte of interest. Aspects of the methods include contacting the saliva sample with an antibacterial agent at some point during the assay, e.g., prior to contact with the test strip device, after contact with the test strip device, etc. By “antibacterial agent” is meant an agent that destroys or inhibits the growth of bacteria, e.g., by killing the bacteria or slowing, including preventing (i.e., arresting), the growth of the bacteria, preventing the respiration of the bacteria which includes the consumption of glucose resident in the saliva sample, etc. As such, antibacterial agents of interest include both bactericidal agents, e.g., agents able to destroy bacteria, and bacteriostatic agents, e.g., agents that arrest the growth or reproduction of bacteria but do not kill bacteria.

Antibacterial agents employed in methods of the invention may vary widely, so long as they exert the desired antibacterial activity and are compatible with the particular signal producing system being employed in the method, e.g., the signal producing system reagents of the test strip device. Antibacterial agents of interest include, but are not limited to: fluoride containing compounds, e.g., sodium fluoride (NaF), SnF₂, sodium monofluorophosphate; tetracyclines, e.g., minocycline, doxycycline, oxytetracycline, etc., rifampin, and norfloxacin, biguanide compounds, triclosan, and benzalkonium chloride, bismuth, cerium, or zinc or silver-containing compounds, e.g., silver salts, including silver salt nanoparticles. Biguanide compounds which may be used according to the invention include poly (hexamethylene biguanide) hydrochloride and chlorhexidine compounds. Chlorhexidine is the term denoting the chemical compound 1,6 bis(N₅-p-chlorophenyl-N₁-biguanido)hexane. Chlorhexidine compounds include chlorhexidine free base (“CHX”) as well as chlorhexidine salts, such as chlorhexidine diphosphanilate, chlorhexidine digluconate (“CHG”), chlorhexidine diacetate (“CHA”), chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine mono-diglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxy-napthoate, and chlorhexidine embonate. Bismuth salts which may be used according to the invention include bismuth nitrate, bismuth citrate, bismuth salicylate, bismuth borate, bismuth mandelate, bismuth palmitate, bismuth benzoate, and bismuth sulfadiazine. Cerium salts which may be used according to the invention include cerium nitrate and other cerium salts having a water solubility similar to cerium nitrate. The term silver-containing compound, as used herein, refers to a compound containing a silver ion unlinked or linked to another molecule via a covalent or noncovalent (e.g., ionic) linkage, including but not limited to covalent compounds such as silver sulfadiazine (“AgSD”) and silver salts such as silver oxide (“Ag₂O”), silver carbonate (“Ag₂CO₃”), silver deoxycholate, silver salicylate, silver iodide, silver nitrate (“AgNO₃”), silver paraaminobenzoate, silver paraaminosalicylate, silver acetylsalicylate, silver ethylenediaminetetraacetic acid (“Ag EDTA”), silver picrate, silver protein, silver citrate, silver lactate and silver laurate. Zinc salts which may be used according to the invention include zinc acetate and other zinc salts having a water solubility similar to zinc acetate. Where desired, the antibacterial agent may be present as a nanoparticle. For example silver compound containing nanoparticles may be employed, where the particles have nanometer dimensions, e.g., ranging from 1 to 1000 nm, such as 2 to 500 nm, e.g., 10 to 250 nm.

In practicing methods of the invention, the antibacterial agent may be employed in the test strip assay of the saliva sample in a number of different ways, so long as the saliva sample contacts the antibacterial agent at some point during the assay, i.e., before the end of the assay. In some instances, the antibacterial agent may be incorporated into the test strip prior to contact of the test strip with the sample. For example, the antibacterial agent may be present in the matrix material of the test strip, e.g., present in or on a bibulous or non-bibulous component of the test strip. In some instances, the antibacterial agent is present in the sample receiving region of the test strip, such that upon application of a volume of sample to the sample receiving region of the test strip, the sample is contacted by the antibacterial agent. In some embodiments, the saliva sample is combined with the antibacterial agent prior to contact of the sample with the test strip. For example, the saliva sample may be contacted with an antibacterial composition, e.g., a composition that includes just the antibacterial agent or the antibacterial agent in combination with one or more additional components, such as a delivery vehicle, buffering agent, etc., to produce an antibacterial agent contacted saliva sample, which is then placed onto the sample receiving region of the test strip. In yet other embodiments, the method includes contacting the test strip with the antimicrobial antibacterial agent after the saliva sample is placed in the sample receiving location. For example, the methods may include spraying the test strip assay device with a liquid volume of the antibacterial agent or placing a liquid drop of the antibacterial agent on to the test strip assay device. A given method may include one or more of the antibacterial agent saliva sample contacting protocols. For example, a saliva sample may be contacted with an antibacterial agent prior to contact with a test strip, where the test strip also includes an amount of antibacterial agent, e.g., present in the sample receiving region of the test strip.

The amount of antibacterial agent that is contacted with the saliva sample may vary as desired, e.g., in view of the particular antibacterial agent, the protocol by which it is contacted with the sample, the nature of the analyte and signal producing system, etc., so long as the amount of antibacterial agent is effective to destroy or inhibit bacteria in the sample to an extent sufficient to obtain suitably accurate results for the assay of interest. In some instances, the amount of antibacterial agent that is contacted with the saliva sample ranges from 0.01 to 3.0 weight %, such as 0.01 to 1.5 weight %, and including 0.01 to 1.0 wt %.

### Test Strip Assay Devices

A variety of different test strips may be employed by the methods of invention, e.g., as described herein. The particular nature of a test strip employed in a given assay will depend on a number of parameters, including but not limited to, the specific analyte to be evaluated, the signal producing system to be employed, etc. Test strips of interest include, but are not limited to, analyte oxidizing signal producing system test strips, lateral flow assay test strips, etc. Non-limiting examples of each of these types of test strips that may be employed in methods of the invention will now be reviewed in greater detail.

### Analyte Oxidation Signal Producing System Reagent Test Strips

Analyte oxidation signal producing reagent test strips include, in some instances, at least the following components: a porous matrix and one or more members of an analyte oxidation signal producing system. The matrix of the test strip may be an inert porous matrix which provides a support for the various members of the signal producing system, described below. The inert porous matrix may be configured to provide a location for physiological sample, e.g., saliva, application (i.e., a sample receiving location) and a location for detection of a product of the signal producing system, e.g., a light-absorbing product or an electron mediator. As such, the inert porous matrix is one that is permissive of aqueous fluid flow through it and provides sufficient void space for the chemical reactions of the signal producing system to take place. A number of different porous matrices have been developed for use in various analyte detection assays, which matrices may differ in terms of materials, pore sizes, dimensions and the like, where representative matrices include those described in: U.S. Pat. Nos. 4,734,360; 4,900,666; 4,935,346; 5,059,394; 5,304,468; 5,306,623; 5,418,142; 5,426,032; 5,515,170; 5,526,120; 5,563,042; 5,620,863; 5,753,429; 5,573,452; 5,780,304; 5,789,255; 5,843,691; 5,846,486; 5,968,836 and 5,972,294. In principle, the nature of the porous matrix is not critical to the subject test strips and therefore is chosen with respect to the other factors, including the nature of the instrument which is used to read the test strip, convenience and the like. As such, the dimensions and porosity of the test strip may vary greatly, where the matrix may or may not have a porosity gradient, e.g. with larger pores near or at the sample application region and smaller pores at the detection region. Materials from which the matrix may be fabricated vary, and include polymers, e.g., polysulfone, polyamides, cellulose or absorbent paper, and the like, where the material may or may not be functionalized to provide for covalent or non-covalent attachment of the various members of the signal producing system, described in greater detail below.

In some embodiments, the subject test strips include a membrane test pad that is affixed to a solid support. The support may be a plastic—e.g., polystyrene, nylon, or polyester—or metallic sheet or any other suitable material known in the art. Associated with the test pad, e.g., coated onto the test pad, incorporated into the test pad, etc., may be a reagent composition. The test strip may also be configured in more complex arrangements, e.g., where the test pad is present between the support and a surface layer, where one or more reagents employed in sample processing may be present on the surface layer. In addition, flow paths or channels may be present on the test strip, as is known in the art.

In the test strips, a dry reagent composition may be associated with, e.g., present on or in, a carrier material or substrate. The carrier material may be bibulous or non-bibulous. By bibulous is meant a material that exhibits preferential retention of one or more components as would occur, for example, in materials capable of absorbing or “imbibing” one or more components, as occurs in chromatographic separations. Examples of bibulous materials include, but are not limited to: nylon, untreated forms of paper, nitrocellulose and the like which result in chromatographic separation of components contained in liquids which are passed therethrough. Alternatively, the substrate may be non-bibulous. Non-bibulous substrates include inert porous matrices which provide a support for the various members of the signal producing system, described infra, and may have a positive charge. These matrices are generally configured to provide a location for the application of a physiological sample, e.g., blood, and detection of the chromogenic product produced by the dye of the signal producing system. As such, the matrix is typically one that is permissive of aqueous fluid flow through it and provides sufficient void space for the chemical reactions of the signal producing system to take place. A number of different porous matrices have been developed for use in various analyte measurement assays, which matrices may differ in terms of materials, pore sizes, dimensions and the like, where representative matrices include those described in U.S. Pat. Nos. 5,932,431; 5,874,099; 5,871,767; 5,869,077; 5,866,322; 5,834,001; 5,800,829; 5,800,828; 5,798,113; 5,670,381; 5,663,054; 5,459,080; 5,459,078; 5,441,894 and 5,212,061; the disclosures of which are herein incorporated by reference. The dimensions and porosity of the test strip may vary greatly, where the matrix may or may not have a porosity gradient, e.g., with larger pores near or at the sample application region and smaller pores at the detection region. In many embodiments, the matrix is configured as a membrane test pad and is affixed to a solid support, where the support may be a plastic (e.g., polystyrene, nylon or polyester) or metallic sheet or any other suitable material known in the art. Of interest are the test strip configurations disclosed in U.S. Pat. Nos. 5,972,294; 5,968,836; 5,968,760; 5,902,731; 5,846,486; 5,843,692; 5,843,691; 5,789,255; 5,780,304; 5,753,452; 5,753,429; 5,736,103; 5,719,034; 5,714,123; 5,620,863; 5,605,837; 5,563,042; 5,526,120; 5,515,170; 5,453,360; 5,426,032; 5,418,142; 5,306,623; 5,304,468; 5,179,005; 5,059,394; 5,049,487; 4,935,346; 4,900,666 and 4,734,360.

In addition to the porous matrix, the subject test strips further include one or more members of a signal producing system which produce a detectable product, e.g., light absorbing product or electron mediator, in response to the presence of an analyte, which detectable product can be used to derive the amount of an analyte present in the assayed sample. In the subject test strips, the one or more members of the signal producing system are associated with, e.g., covalently or non-covalently attached to, at least a portion of (e.g., the detection region) the porous matrix, including substantially all, if not all, of the porous matrix.

As indicated above, in these types of test strips, the signal producing system is an analyte oxidation signal producing system. By analyte oxidation signal producing system is meant that in generating the detectable signal from which the analyte concentration in the sample is derived, the analyte is oxidized by a suitable enzyme to produce a detectable product, e.g., a light absorbing compound (e.g., as employed in colorimetric test strips) or an enzyme mediator (e.g., as employed in electrochemical test strips).

In one type of a colorimetric test strip of interest that may be employed in methods of the invention, the analyte is oxidized by a suitable enzyme to produce an oxidized form of the analyte and a corresponding or proportional amount of hydrogen peroxide. The hydrogen peroxide is then employed, in turn, to generate the detectable product from one or more indicator compounds, where the amount of detectable product produced by the signal producing system, i.e., the signal, is then related to the amount of the analyte in the initial sample. As such, the analyte oxidation signal producing systems present in the subject test strips are also correctly characterized as hydrogen peroxide based signal producing systems.

As indicated above, the hydrogen peroxide based signal producing systems include an enzyme that oxidizes the analyte and produces a corresponding amount of hydrogen peroxide, where by corresponding amount is meant that the amount of hydrogen peroxide that is produced is proportional to the amount of the analyte present in the sample. The specific nature of this first enzyme necessarily depends on the nature of the analyte being assayed but is generally an oxidase. As such, the first enzyme may be: glucose oxidase (where the analyte is glucose). In those embodiments where the reagent test strip is designed for the detection of glucose concentration, the first enzyme may be glucose oxidase. The glucose oxidase may be obtained from any convenient source, e.g., a naturally occurring source such as Aspergillus niger, or recombinantly produced.

In some embodiments, the subject signal producing systems also include an enzyme cofactor that is capable of interacting with the oxidizing agent in a manner such that the analyte of interest is oxidized by the oxidizing agent, which agent concomitantly reduces the enzyme cofactor. Enzyme cofactors of interest include, but are not limited to: beta-nicotinamide adenine dinucleotide (beta-AND); beta-nicotinamide adenine dinucleotide phosphate (beta-NADP); thionicotinamide adenine dinucleotide; thionicotinamide adenine dinucleotide phosphate; nicotinamide 1,N6-ethenoadenine dinucleotide; nicotinamide 1,N6-ethenoadenine dinucleotide phosphate; and pyrrolo-quinoline quinone (PQQ); and flavin compounds, such as FAD and FMN. Enzyme cofactors of interest that may be included in the subject signal producing systems include: NADH or AND(P)H and PQQH2.

Signal producing systems may include a second enzyme. The second enzyme of the signal producing system, when present, may be an enzyme that catalyzes the conversion of one or more indicator compounds into a detectable product in the presence of hydrogen peroxide, where the amount of detectable product that is produced by this reaction is proportional to the amount of hydrogen peroxide that is present. This second enzyme may be a peroxidase, where suitable peroxidases include: horseradish peroxidase (HRP), soy peroxidase, recombinantly produced peroxidases and synthetic analogs having peroxidative activity and the like. See e.g., Y. Ci, F. Wang; Analytica Chimica Acta, 233 (1990): 299-302.

The indicator compound or compounds, e.g., substrates, are ones that are either formed or decomposed by the hydrogen peroxide in the presence of the peroxidase to produce an indicator dye that absorbs light in a predetermined wavelength range. In some instances, the indicator dye absorbs strongly at a wavelength different from that at which the sample or the testing reagent absorbs strongly. The oxidized form of the indicator may be the colored, faintly-colored, or colorless final product that evidences a change in color of the testing side of the membrane. That is to say, the testing reagent can indicate the presence of glucose in a sample by a colored area being bleached or, alternatively, by a colorless area developing color.

Indicator compounds that are useful in the present invention include both one- and two-component chromogenic substrates. One-component systems include aromatic amines, aromatic alcohols, azines, and benzidines, such as tetramethyl benzidine-HCl. Suitable two-component systems include those in which one component is MBTH, an MBTH derivative (see for example those disclosed in U.S. Pat. No. 5,563,031), or 4-aminoantipyrine and the other component is an aromatic amine, aromatic alcohol, conjugated amine, conjugated alcohol or aromatic or aliphatic aldehyde. Exemplary two-component systems are 3-methyl-2-benzothiazolinone hydrazone hydrochloride (MBTH) combined with 3-dimethylaminobenzoic acid (DMAB); MBTH combined with 3,5-dichloro-2-hydroxybenzene-sulfonic acid (DCHBS); and 3-methyl-2-benzothiazolinone hydrazone N-sulfonyl benzenesulfonate monosodium (MBTHSB) combined with 8-anilino-1 naphthalene sulfonic acid ammonium (ANS). In certain embodiments, the dye couple MBTHSB-ANS is preferred. In yet other embodiments, signal producing systems that produce a fluorescent detectable product (or detectable non-fluorescent substance, e.g. in a fluorescent background) may be employed, such as those described in: Kiyoshi Zaitsu, Yosuke Ohkura: New fluorogenic substrates for Horseradish Peroxidase: rapid and sensitive assay for hydrogen peroxide and the Peroxidase. Analytical Biochemistry (1980) 109, 109-113.

Examples of colorimetric test strips that may be employed in methods of the invention are further described in U.S. Pat. Nos. 3,964,871; 4,269,938; 5,418,142; 5,620,863; 5,789,255; 5,843,691; 5,843,692; 5,843,691; 5,843,692; 6,485,923; 6,656,697; 6,984,307; 7,112,265; the disclosure of which is herein incorporated by reference.

In electrochemical test strips, reagent compositions of interest include an enzyme component and a redox mediator (electron transfer mediator). The enzyme component may be an enzyme or plurality of enzymes that work in concert to oxidize the analyte of interest. In other words, the enzyme member may be made up of a single analyte oxidizing enzyme or a collection of two or more enzymes that work in concert to oxidize the analyte of interest, allowing generation of the electrochemical signal detected. Enzymes of interest include oxidases, dehydrogenases, lipases, kinases, diaphorases, quinoproteins and the like. The enzyme selected in the reaction depends on the particular analyte for which the electrochemical test strip comprising the enzyme is designed to detect. Representative enzymes include: glucose oxidase, glucose dehydrogenase, glycerol kinase, glycerol-3-phosphate oxidase, lactate oxidase, lactate dehydrogenase, pyruvate oxidase, alcohol oxidase, bilirubin oxidase, and the like.

Another component of the reagent composition is a redox mediator, which may comprise one or more mediator agents. The mediator acts as an intermediary that facilitates the transfer of electrons from the enzyme (which has taken one or more electrons from the analyte during analyte oxidation) to an electrode, e.g., which may be incorporated into the test strip. A variety of different mediator agents known in the art may be used, including ferricyanide, phenazine ethosulphate, phenazine methosulfate, phenylenediamine, N,N,N′,N′-tetramethyl phenylenediamine, 1-methoxy-phenazine methosulfate, 2,5-dimethyl-1,4-benzoquinone, 2,6-dimethyl-1,4-benzoquinone, 2,5-dichloro-1,4-benzoquinone, ferrocene derivatives, osmium bipyridyl complexes, ruthenium complexes and the like. In many embodiments, the redox mediator is ferricyanide. Other reagents that may be present in the reaction area include buffering agents, (e.g., citraconate, citrate, phosphate), “Good” buffers and the like.

In addition, electrochemical test strips of interest may include one or more electrode components and related circuitry which are configured to detect a redox mediator and transfer an electronic signal resulting from contact therefrom with the electrode to a suitable meter.

Examples of electronic test strips that may be employed in methods of the invention are further described in U.S. Pat. Nos. 8,758,582; 8,702,960; RE43,815; RE42,924; 8,057,659; RE42,560; RE41,309; 7,653,492; 7,498,132; 7,419,573; 7,387,714; 7,063,776; 6,863,800; 6,855,243; 6,716,577; 6,558,528; and 6,270,637; the disclosure of which is herein incorporated by reference.

### Lateral Flow Assay Test Strips

Another type of test strip that may be employed in methods of the invention is a lateral flow assay test strip. As these assay devices are “lateral flow” assay devices, they are configured to receive a sample of interest at a sample receiving region and to provide for the sample to move laterally through a bibulous material (i.e., bibulous member) by capillary action to a detection region, such that the sample is wicked laterally through the bibulous member from the sample receiving region to the detection region.

Bibulous members of devices of the invention may be fabricated from any convenient material, e.g., as described above. Examples of bibulous materials of interest include, but are not limited to: organic or inorganic polymers, and natural and synthetic polymers. More specific examples of suitable solid supports include, without limitation, glass fiber, cellulose nylon, crosslinked dextran, various chromatographic papers and nitrocellulose.

While the bibulous member and overall configuration of the lateral assay device may vary, in certain embodiments the bibulous member has a strip configuration. Where the bibulous material is configured as a strip, the bibulous member has a length that is longer than its width. While any practical configuration may be employed, in some instances the length is longer than the width by 1.5 fold or more, such as 2-fold or more, e.g., 10 fold or more, including 20-fold or more. In some instances, the length of the bibulous member ranges from 0.5 to 20 cm, such as 1.0 to 15 cm, e.g., 2.0 to 10 cm, while the width ranges 0.1 to 5.0 cm, such as 0.5 to 2.5 cm, e.g., 1 to 2 cm. The thickness of the bibulous member may also vary, ranging in some instances from 0.01 to 0.05 cm, such as 0.1 to 0.4 cm, e.g., 0.1 to 0.25 cm.

In addition to the bibulous member, lateral flow assay devices may include a sample receiving region. The sample receiving region may simply be a first region of the bibulous member, e.g., positioned closer to one end of the bibulous member. Alternatively, the sample receiving region may be distinct from the bibulous member, but configured to provide for fluid communication of sample into the bibulous member upon application of sample to the sample receiving region. The sample receiving region may be configured to receive samples of varying volumes, where in some instances the sample receiving region is configured to receive a sample having a volume ranging from 0.1 to 1000 μl, such as 5 to 20 μl and including 50 to 200 μl. In some instances, the sample receiving region may include a metering device configured to meter a specific amount of sample into the bibulous member. Examples of metering devices of interest include those described in United States Published Patent Application Nos.: 20080145272; 20070134810; 20060008847; and 20050227370.

In addition to the sample receiving region, lateral flow assay devices of the invention further include a detection region. A detection region is a region of the bibulous member from which a result may be read during use of the device. The detection region is positioned at some distance downstream from the sample receiving region of the device. By “downstream” is meant the lateral direction that the sample flows by capillary action, i.e., the direction of fluid flow from the sample receiving region. The distance between the sample receiving region and the detection region may vary, ranging in some instances from 0.3 to 15 cm, such as 1 to 15 cm and including 5 to 10 cm, e.g., 1 to 5 cm.

The detection region is a region that includes at least one distinct capture probe region. The capture probe region is a region that includes an amount of capture probe stably associated with the bibulous member in the capture probe region. The size of the capture probe region may vary, and in some instances the capture probe region has an area ranging from 0.01 to 0.5 cm², such as 0.05 to 0.1 cm² and including 0.1 to 0.2 cm². The capture probe region may have a variety of different configurations, where the configuration may be a line, circle, square, or more complex shape, such as a “+”, as desired.

As indicated above, the capture probe region includes a capture probe stably associated with the bibulous material of the bibulous member. By “stably associated with” is meant that the capture probe and the bibulous member maintain their position relative to each other in space under the conditions of use, e.g., under the assay conditions. As such, the capture probe and the bibulous member can be non-covalently or covalently stably associated with each other. Examples of non-covalent association include non-specific adsorption, binding based on electrostatic interactions (e.g., ion-ion pair interactions), hydrophobic interactions, hydrogen bonding interactions, and the like. Examples of covalent binding include covalent bonds formed between the capture probe and a functional group present on the bibulous material.

Capture probes are molecules that specifically bind to an analyte of interest. The terms “specific binding,” “specifically bind,” and the like, refer to the ability of the capture probe to preferentially bind directly to the analyte of interest relative to other molecules or moieties in a solution or reaction mixture that may be present in the bibulous member. In certain embodiments, the affinity between a capture probe and the analyte to which it specifically binds when they are specifically bound to each other in a binding complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M.

A variety of different types of specific binding agents may be employed as the capture probe. Specific binding agents of interest include antibody binding agents, proteins, peptides, haptens, nucleic acids, etc. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′₂ fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

A given detection region may include a single capture probe region or two or more different capture probe regions, where each of the two or more different capture probe regions includes a capture probe, where the capture probe in each region may be the same (such as is found in the quantitative assay devices as described in greater detail below) or different (such as may be present in multiplex assay devices as described in greater detail below). Where the detection region includes two or more capture probe regions, the regions may be distinct from each other or overlapping, as desired.

In some instances, the bibulous member may include a reporter binding member positioned upstream from the detection region, e.g., either in the sample receiving region or a location between the sample receiving region and the detection region. The distance between the reporter binding member and the detection region may vary, ranging in some instances from 0.3 to 15 cm, such as 1 to 5 cm and including 5 to 10 cm. The reporter binding member, when present, is non-stably associated with the bibulous member. By “non-stably associated” is meant that while the reporter binding member may be stationary relative to the bibulous member prior to sample application, upon sample application and sample wicking through the bibulous binding member, the reporter binding member is free to react with the analyte present in the sample and to move with the sample through the bibulous member by capillary action. As such, the reporter binding member moves laterally through the bibulous member under the bulk fluid flow forces.

Reporter binding members of interest include a specific binding member and a signal producing system member. In the reporter binding member, the specific binding member and the signal producing system member are stably associated with each other, e.g., via covalent bonding.

The specific binding member may vary depending on whether the assay has a competitive or sandwich format. For competitive formats, the binding member is a moiety that competes with the analyte of interest for binding to the capture probe in the detection region. The binding member may be the analyte or a fragment thereof. For sandwich formats, the binding member specifically binds to the analyte at a location that is different from the location to which the capture probe binds. As such, the binding member and the capture probe may simultaneously bind to the analyte of interest. In these sandwich formats, the analyte specific binding moiety may be any moiety that specifically binds to the analyte of interest. Specific binding members of interest include antibody binding members, proteins, peptides, haptens, nucleic acids, etc. The term “antibody binding member” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′₂ fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

In addition to the binding member, the reporter binding member further includes a member of a signal producing system. The member of the signal producing system may vary widely depending on the particular nature of the lateral flow assay and may be any directly or indirectly detectable label. Suitable detectable labels for use in the above methods include any moiety that is detectable by spectroscopic, photochemical, biochemical, immunochemical, electrical, optical, chemical, or other means. For example, suitable labels include biotin for staining with labeled streptavidin conjugate, fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex beads). Patents that describe the use of such labels include U.S. Pat. Nos. 3,817,837; 3,850,752; 3,939,350; 3,996,345; 4,277,437; 4,275,149; and 4,366,241. See also Handbook of Fluorescent Probes and Research Chemicals (6th Ed., Molecular Probes, Inc., Eugene, Oreg.). Radiolabels can be detected using photographic film or scintillation counters. Fluorescent markers can be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

In some instances, the lateral flow assay device may further include a control region. The control region is located downstream from the sample receiving region, and may be located upstream or downstream from the detection region, as desired. The control region contains immobilized control agents. The immobilized control agents bind specifically to mobile control binding agents to form a control binding pair, e.g., as described in U.S. Pat. No. 6,136,610. Control binding pairs of interest act as internal controls, that is, the control against which the analyte measurement results may be compared on the individual test strip. Although, in general, any conventional controls can be used herein, in some instances control compounds that do not exist in the sample or do not immunologically cross-react with compounds that exist in the sample are employed. Examples of suitable control binding pairs of interest include, but are not limited to: Mouse IgG/anti-mouse IgG, chicken IgY/anti-chicken IgY, etc. Either member of these pairs may be the immobilized control agent, with the other being the control binding agent. A given lateral flow assay device may have a single control region or two or more different control regions, where the immobilized control agents of each region may be the same or different. The control binding agent may optionally be non-stably associated with the bibulous member at a location that is upstream from the control region, e.g., at a location that is the same as or different from the reporter binding agent.

Optionally, the lateral flow assay device may include an absorbent pad downstream from the detection region and any control region, e.g., at the end distal from the sample receiving region, where the absorbent pad is configured to absorb fluid and reagents present therein that have flowed through the bibulous member.

Where desired, the component parts of the lateral flow assay device may be present in a suitable housing. The housing may be configured to enclose the bibulous member and other assay components. The housing may be fabricated from any suitable material, where the material may be a material that is sufficiently rigid to maintain the integrity of the bibulous member and other components housed therein and also inert to the various fluids and reagents that contact the housing during use. Housing materials of interest include plastics. The housing may include a port or analogous structure configured to allow sample application to the sample application region and a window configured to allow viewing of the detection region. The housing may further include markings, e.g., detection region and control region markings (e.g., “T” and “C”), etc.

### Sample Application and Analyte Detection

As summarized above, in practicing methods of the invention, a saliva sample is positioned onto a sample receiving region of a test strip, where the saliva sample is contacted with an antibacterial agent at some point during the assay, e.g., as described above. In practicing embodiments of the methods, a quantity of the saliva sample is applied to the test strip. The amount of saliva sample that is applied to the test strip may vary. In some instances, the amount of saliva that is contacted with the test strip ranges from 1 to 500 microliters of saliva, such as 1 to 100 microliters of saliva and including 1 to 10 microliters of saliva.

Following sample application, the test strip may be maintained for a period of time, e.g., a sample processing time (e.g., sample incubation time), and then a signal may be obtained from the test strip. The sample processing time, e.g., incubation time, may vary, and in some instances ranges from 1 second to 1 hour, such as 5 seconds to 30 minutes, e.g., 10 seconds to 10 minutes.

Following the sample processing time, a signal is obtained from the test strip and employed to determine the presence of the analyte in the saliva sample. The determination of the presence of the analyte may be qualitative or quantitative, as desired. Accordingly, the above described methods of detecting the presence of an analyte in a saliva sample find use in a variety of different applications.

The signal may be obtained and processed using any convenient device or protocol, where in some instances the signal is obtained and processed to obtain a result that includes information about the presence of the analyte, e.g., either quantitative or qualitative, in the sample by using a device or meter configured to do so. For example, colorimetric or electrochemical test strip meters may be employed as desired, where such meters include, but are not limited to, those described in U.S. Pat. Nos. 8,758,582; 8,702,960; RE43,815; RE42,924; 8,057,659; RE42,560; RE41,309; 7,653,492; 7,498,132; 7,419,573; 7,387,714; 7,112,265; 7,063,776; 6,984,307; 6,863,800; 6,855,243; 6,716,577; 6,656,697; 6,558,528; 6,485,923; 6,270,637; 5,843,692; 5,843,691; 5,789,255; 5,620,863; 5,418,142; 4,269,938; and 3,964,871; the disclosure of which is herein incorporated by reference.

### Utility

The above described methods and compositions find use in a variety of applications, including applications where it is desired to assay a saliva sample for an analyte. The subject methods may be used to screen a saliva sample for the presence or absence of one or more analytes in the sample. As indicated above, the method may be qualitative or quantitative. As such, where detection is qualitative, the methods provide a reading or evaluation, e.g., assessment, of whether or not a target analyte is present in the sample being assayed. In yet other embodiments, the methods provide a quantitative detection of whether the target analyte is present in the sample being assayed, i.e., an evaluation or assessment of the actual amount of the target analyte in the sample being assayed. In such embodiments, the quantitative detection may be absolute or, if the method is a method of detecting two or more different target analytes in a sample, relative. As such, the term “quantifying” when used in the context of quantifying a target analyte(s) in a sample can refer to absolute or to relative quantification.

The methods and compositions described herein may be employed to assay a sample, such as a saliva sample, for a variety of different analytes, where analytes of interest include, but are not limited to: glucose, cortisol, melatonin, sex hormones, e.g., estradiol, progesterone, luteinizing hormone, dehydroepiandrosterone (DHEA), and testosterone; neoplastic condition markers, e.g., pancreatic condition markers (such as mRNA biomarkers), breast cancer condition markers (such as CA15-3 and P53), oral cancer markers (such as transferrin, cyclin D1, maspin, and mRNAs; infectious condition analytes, such as anti-HIV antibody, HBV surface antigen, etc., and chemical substances, including substances of abuse.

Saliva samples may be obtained from any convenient source. In certain embodiments, the saliva sample is one that is obtained from a “mammal” or “mammalian subject”, where these terms are used broadly to describe organisms which are within the class Mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult.

### Kits

Aspects of the invention further include kits, where kits include one or more test strips and an antibacterial agent, e.g., as described above, where the antibacterial agent may be part of the test strip or separate from the test strip, depending on the particular protocol for which the kit is configured. In some embodiments, devices of the kits further include one or more assay components (e.g., a competitor, a reporter, a mobile control binding agent, and the like). Any assay component can be included as part of a test strip assay device or can be included in a kit separate from the test strip assay device. As such, in addition to a test strip assay device, kits can include one or more assay components (e.g., a competitor, a reporter, a mobile control binding agent, a buffer, a reagent for dilution, a reagent for reconstitution, a sample applicator, and the like). The various assay components of the kits may be present in separate containers, or some or all of them may be pre-combined into a reagent mixture.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

The following examples are offered by way of illustration and not by way of limitation.

### EXPERIMENTAL

Individual unstimulated saliva samples were harvested into separate glass vials dedicated to each of 10 study participants. Within no more than one minute after harvesting, a saliva sample was deposited onto two identical strip assays capable of quantitative measurement of glucose concentration.

Within 15 seconds of saliva sample deposition, a drop of 0.1 wt % NaF solution was placed onto one of the two deposited saliva samples. As a control, no NaF solution was deposited onto one of the two deposited saliva samples. Glucose measurements from the strip assays were recorded. As a study reference, glucose concentrations of the harvested saliva samples were measured within 30 seconds of harvesting using a high performance liquid chromatography (HPLC) instrument.

Three separate unstimulated saliva samples were harvested from each study participant within five minutes of each other. Glucose measurements were made as described above. Glucose concentrations are reported as an average of the three harvested sample measurements.

Comparison of the matched pairs of glucose measurements (with and without NaF solution deposition) was done. Glucose measurements made with the deposition of NaF solution show higher percent mean average relative difference to the glucose concentration made with the HPLC method. Glucose measurement made without the deposition of NaF solution show lower percent mean average relative difference to the glucose concentration made with the HPLC method.

Notwithstanding the appended clauses, the disclosure set forth herein is also defined by the following clauses:

1. A method of evaluating a sample for the presence of an analyte, the method comprising:

a) placing the sample onto a sample receiving location of a test strip assay device comprising analyte detection reagents; and

b) obtaining a signal from the test strip assay device to evaluate the sample for the presence of the analyte;

wherein the method comprises contacting the saliva sample with an antibacterial agent.

2. The method according to Clause 1, wherein the test strip assay device comprises the antibacterial agent.
3. The method according to Clause 2, wherein the antibacterial agent is present in the sample receiving location of the test strip assay device.
4. The method according to Clause 1, wherein the method comprises contacting the sample with the antibacterial agent prior to placing the sample onto the sample receiving location.
5. The method according to Clause 1, wherein the method comprises contacting the test strip assay device with the antibacterial agent after the sample is placed in the sample receiving location.
6. The method according to Clause 5, wherein the contacting comprises spraying the test strip assay device with the antibacterial agent.
7. The method according to Clause 5, wherein the contacting comprises placing a liquid drop of the antibacterial agent onto the test strip assay device.
8. The method according to any of the preceding clauses, wherein the antibacterial agent is a bactericidal agent.
9. The method according to any of the preceding clauses, wherein the antibacterial agent is a bacteriostatic agent.
10. The method according to any of the preceding clauses, wherein the antibacterial agent is selected from the group consisting of: sodium fluoride, triclosan, silver salt particles and combinations thereof.
11. The method according to Clause 10, wherein the silver salt particles are silver salt nanoparticles.
12. The method according to any of the preceding clauses, wherein the sample is a human sample.
13. The method according to Clause 12, wherein the sample is a saliva sample.
14. The method according to any of the preceding clauses, wherein the analyte is glucose.
15. The method according to any of the preceding clauses, wherein the analyte detection reagents comprise analyte oxidation signal producing reagents.
16. The method according to any of the preceding clauses, wherein the evaluating is qualitative.
17. The method according to any of the preceding clauses, wherein the evaluating is quantitative.
18. A test strip assay device, the device comprising:

analyte detection reagents; and

an antibacterial agent.

19. The device according to Clause 18, wherein the antibacterial agent is present in the sample receiving location of the test strip assay device.
20. The device according to any of Clauses 18 and 19, wherein the antibacterial agent is a bactericidal agent.
21. The device according to any of Clauses 18 and 20, wherein the antibacterial agent is a bacteriostatic agent.
22. The device according to any of Clauses 18 to 21, wherein the antibacterial agent is selected from the group consisting of: sodium fluoride, triclosan, silver salts and combinations thereof.
23. The device according to Clause 22, wherein the silver salt particles are silver salt nanoparticles.
24. The device according to any of Clauses 18 to 23, wherein the analyte is glucose.
25. The device according to any of Clauses 18 to 24, wherein the analyte detection reagents comprise analyte oxidation signal producing reagents.
26. A kit comprising:

a test strip assay device comprising analyte detection reagents; and

an antibacterial agent.

27. The kit according to Clause 26, wherein the test strip assay device comprises the antibacterial agent.
28. The kit according to Clause 27, wherein the antibacterial agent is present in the sample receiving location of the test strip assay device.
29. The kit according to Clause 28, wherein the antibacterial agent is separate from the test strip assay device.
30. The kit according to any of Clauses 26 to 29, wherein the antibacterial agent is a bactericidal agent.
31. The kit according to any of Clauses 26 to 30, wherein the antibacterial agent is a bacteriostatic agent.
32. The kit according to any of the preceding clauses, wherein the antibacterial agent is selected from the group consisting of: sodium fluoride, triclosan, silver salt particles and combinations thereof.
33. The kit according to Clause 32, wherein the silver salt particles are silver salt nanoparticles.
34. The kit according to any of Clauses 26 to 33, wherein the analyte is glucose.
35. The kit according to any of Clauses 26 to 34, wherein the analyte detection reagents comprise analyte oxidation signal producing reagents.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof.

Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of evaluating a sample for the presence of an analyte, the method comprising:
a) placing the sample onto a sample receiving location of a test strip assay device comprising analyte detection reagents; and
b) obtaining a signal from the test strip assay device to evaluate the sample for the presence of the analyte;
wherein the method comprises contacting the saliva sample with an antibacterial agent.

2. The method according to claim 1, wherein the test strip assay device comprises the antibacterial agent.

3. The method according to claim 2, wherein the antibacterial agent is present in the sample receiving location of the test strip assay device.

4. The method according to claim 1, wherein the method comprises contacting the sample with the antibacterial agent prior to placing the sample onto the sample receiving location.

5. The method according to claim 1, wherein the method comprises contacting the test strip assay device with the antibacterial agent after the sample is placed in the sample receiving location.

6. The method according to claim 5, wherein the contacting comprises spraying the test strip assay device with the antibacterial agent or placing a liquid drop of the antibacterial agent onto the test strip assay device.

7. The method according to any of the preceding claims, wherein the antibacterial agent is a bactericidal agent or a bacteriostatic agent.

8. The method according to any of the preceding claims, wherein the sample is a human sample.

9. The method according to claim 8, wherein the sample is a saliva sample.

10. The method according to any of the preceding claims, wherein the analyte is glucose.

11. A test strip assay device, the device comprising:
analyte detection reagents; and
an antibacterial agent.

12. The device according to claim 11, wherein the antibacterial agent is present in the sample receiving location of the test strip assay device.

13. A kit comprising:
a test strip assay device comprising analyte detection reagents; and
an antibacterial agent.

14. The kit according to claim 13, wherein the test strip assay device comprises the antibacterial agent.

15. The kit according to claim 13, wherein the antibacterial agent is separate from the test strip assay device.
